(19) United States
(12) Patent Application Publication
(10) Pub. No.: US 2005/0176027 A1
(43) Pub. Date: Aug. 11, 2005

(54) Nucleic acid extraction method

(76) Inventors: Joseph G. Suzow, Monroeville, PA (US); Zhili Lin, Sewickley, PA (US); Jamie M. Fontaine, Cranberry Township, PA (US)

Correspondence Address: Kathy Smith Dias, Esq.;HESLIN ROTHENBERG FARLEY & MESITI P.C., 5 Columbia Circle, Albany, NY, 12203-5160, US

(21) Appl. No.: 10/959,770

(22) Filed: Oct. 6, 2004

Related U.S. Application Data
(60) Provisional application No. 60/510,880, filed on Oct. 14, 2003.

(51) Int. Cl. C12Q 1/68; C12N 1/08

(52) U.S. Cl. 435/6; 435/270

(57) ABSTRACT

A method for extracting DNA from a specimen is provided which is cost-efficient, nontoxic to laboratory workers and is automation-compatible to meet high-throughput requirements in newborn screening and other nucleic acid applications. Methanol is added and evaporated at a high temperature to ensure the heme and other large proteins bind to the filter paper thus preventing them from going into solution during extraction and inhibiting later PCR reactions. A buffer and salt concentration is then added to each specimen to continue to bind the heme protein to the filter paper when the DNA is extracted from the filter paper at an optimal pH. The plate is then heated to release the DNA into the buffer without releasing excess heme protein which may inhibit PCR reactions.

### SPECIFIC REFERENCE

This application hereby claims benefit of provisional application Ser. No. 60/510,880, filed Oct. 14, 2003.

### BACKGROUND

1. Field of the Invention

The present invention relates to automation-compatible, low cost, and low human toxicity methods for the extraction of nucleic acids from blood spotted on a matrix. Particularly, what is disclosed is a protocol developed to meet the requirements of a high throughput newborn screening laboratory and other clinical or forensic labs using a combination of reagents and heating techniques. The assays are useful for making reagents and consumables economically feasible for high throughput, enhancing automation compatibility, and lowering reagent human toxicity.

2. Description of the Related Art

Specific techniques for extracting DNA from a paper matrix are known in the art. Generally, the current technology involves the extraction of DNA from a cellulose filter card having spotted thereon one or more blood drops. In these methods, either the DNA is extracted from the card and put into solution, or the DNA is left on the paper matrix and a piece of the paper matrix is used for setting up assays.

U.S. Pat. No. 6,410,725 to Scholl et al. for example, describes a method of extracting DNA from dried biological samples on solid substrates. A DNA extraction solution containing, in part, formamide, citrate and a buffer contacts the biological sample. The resultant mixture is heated, yielding a supernatant containing the DNA, which is then isolated. The combination of chemicals and reagents in the solution allow for the extraction, and concurrently, the removal or inactivation of the compounds present in the paper matrix.

Drawbacks in the above and other prior methods exist, e.g., excess amounts of buffer usage, multiple step of buffer usage and/or the need to use additional chemicals in the reagents dramatically increase the cost of the assay.

Additional limitations are that prior methods are logistically difficult, cost prohibitive, and contain hazardous compounds which are not compatible with automation and high throughput screening applications and environments. As an example formamide is highly toxic, so in a high-throughput clinical environment, its use is both costly and potentially unsafe. In the automation and high-throughput process, the majority of expenses are incurred from costs associated with these reagents and required consumables, especially when excess or alternative reagents must be used to remove or inactivate compounds present on certain types of filter paper.

Furthermore, the number of pipet tips used for liquid transfer from one position to another may become excessive. Most prior art and commercial extraction methods require a significant number of liquid transfers to complete the nucleic acid extraction. This large number of reagent components and liquid transfers results in high pipet tip consumption and high automation cost.

In addition to the limitations imposed by high costs, the method must also accomodate established automation techniques. Particularly, current methods are incompatible with standard automation techniques involving extraction from specimens dried on a solid matrix. Automation incompatibility issues arise when the composition of extraction reagents cause the unused dried blood specimen (DBS) to be removed from its vessel and unintentionally or accidentally discarded by the automation system. This discarding of the unused DBS can be attributed to the concentrations of salts and other compounds dissolved in the extraction reagents. If the reagent is not of specific concentration and composition, the DBS floats on the reagent surface when the reagent is added to the DBS. While the DBS is floating, one must use the automated pipet tips to transfer liquid into and out of the vessel containing the DBS. This transfer of liquid has been shown to be inadequate because the floating DBS has a tendency to cling to the pipet tip during liquid transfer. When the pipet tip is lifted from the vessel, the unused DBS remains attached to the pipet tip and is immediately discarded as the automation system discards the pipet tip. The DBS is then lost and can not be recovered. This is not acceptable for a clinical procedure due to its inherent waste.

Furthermore, in addition to automation compatibility, the types of reagents used often governs a process. For example, although a variety of reagents can be used for efficient nucleic acid extraction, many of these are not compatible with the assays which will utilize the extracts. This is of particular concern in a high throughput application where very small assay reaction volumes are often necessary to maximize throughput. Because of this small reaction volume, the volume of the extract constitutes a significant proportion of the total reaction volume. Therefore, the reagent used for extraction can significantly influence the assay reaction conditions. In order to address this concern, the present method was developed to allow for the use of reagents that are optimally compatible with, and easily adaptable to, a variety of assay reactions.

### SUMMARY OF THE INVENTION

It is an objective of the present invention to provide a method for nucleic acid extraction wherein methanol alone is used to fix the heme protein to the blood card before the extraction process begins.

It is further an objective to use a Tris buffer or similar buffer alone for the step of extraction in the absence of other additional chemicals.

It is further an objective of the present invention to provide a method using only reagents which allow the assay to be more automation-compatible by eliminating specimen centrifugation steps and additionally preventing the loss of the specimen during liquid pipeting.

It is further an objective to use reagents that are nontoxic or which are, at least, minimally toxic.

What is provided is a method for extracting nucleic acid from a paper matrix, wherein the first step involves generally fixing the blood heme protein and other proteins to the paper matrix using an alcohol solution and heating step. This step is necessary because proteins often inhibit or interfere with nucleic acid assay reactions. Differing from this approach, most other nucleic acid extraction methods attempt to extract the proteins out of the paper matrix. A second innovation used to keep the protein attached to the paper matrix is the use of a reagent concentration which is favorable to maintaining protein attached to the matrix. If the reagent concentration is too low, protein will release from the matrix and inhibit downstream applications. Finally, a third reagent innovation is the use of a reagent which buffers the pH of the extracted specimen in order to maintain a pH optimal for the assay in which it would be used. The pH of the extraction buffer is a critical factor for use in low reaction volume high throughput assay reactions. A type of buffer is used that is readily adjustable to a variety of pH values which will suit the desired assay conditions. This pH buffer contributes additional functions necessary to the success of the extraction. In addition to stabilizing pH, the buffer plays a key role in ensuring the adherence of protein to the paper matrix and preventing the DBS from sticking to, and being discarded with, the pipet tips during automated pipeting.

Accordingly, the method comprises the steps of punching a blood spot into each well of a well plate; adding MetOH (methyl alcohol, methanol) to each specimen; evaporating the methanol, wherein the heme and other large proteins bind to the filter paper thus preventing them from going into solution during extraction and inhibiting later PCR or other reactions; adding Tris-HCl buffer to each specimen, such that the DNA is extracted from the filter paper, a reagent concentration is provided to prevent heme protein from coming out of the paper, and the sample is buffered to the correct pH needed for later PCR or other reactions; sealing the plate with a strong heat sealing device; and heating the plate. This step releases the DNA into the Tris-Buffer without releasing excess heme protein which may inhibit PCR reactions.

### DETAILED DESCRIPTION OF THE PREFERRED EMBODIMENT

The invention will now be described in detail in relation to a preferred embodiment and implementation thereof which is exemplary in nature and descriptively specific as disclosed. As is customary, it will be understood that no limitation of the scope of the invention is thereby intended. The invention encompasses such alterations and further modifications in the illustrated method, and such further applications of the principles of the invention illustrated herein, as would normally occur to persons skilled in the art to which the invention relates.

### DEFINITIONS

Reagent—any individual or mixture of liquids or solids which is used to allow the extraction.

Dried Blood Spot (DBS)—one or more drops of blood placed and dried on a paper matrix or other solid substrate.

Pipet—a mechanical device used to transfer liquid from one location to another by siphoning action.

Pipet tips—a plastic sheath used to cover the ends of the liquid transfer device (pipet) wherein liquid is taken into or expelled from in order to transfer a liquid from one location to another.

Extraction Vessel—any piece of standard labware used to contain specimens during specimen manipulation.

Nucleic Acid—any molecule consisting of linked nucleic or ribonucleic acids (DNA, RNA etc.).

Solid Substrate—any solid substrate used as a medium for collecting and/or storing blood specimens.

Assay—any chemical or biological reaction used to achieve the detection or measurement of a biological, physical, or chemical process or entity.

Though the techniques for the current procedures may be performed manually, the current process lends itself to an automation process having generally three parts: 1) the transfer of liquids from one position to another; 2) the transfer of labware from one position to another; and 3) the use of automation system software to coordinate the sequence, timing, and liquid volumes for liquid and labware movement.

The automation system transfers liquid by picking up the liquid by air displacement or liquid displacement, similar to a syringe. The mechanical part of the instrument then moves the liquid to the next position and dispenses it. For current automation technology used for high-throughput applications, the pipetor can pick up or dispense up to 96 liquids at once. Automation systems which transfer 384, or more or less liquids, are common. 8, 96, 384, and 1536 tip pipeting devices are commonly known in the art. An example of liquid transfer is the pick up of the MetOH from a single large well container and dispensing the liquid into each of the 96 wells of a microtiter plate.

The system may also move labware with mechanical arms and grippers. An example for the current method is the pick up of the 96-well plate containing the MetOH and placement on a heating block for incubation.

All of the liquid and labware transfer is preferably coordinated using software such as that written by Beckman Coulter for its automation system. Items that are set up in the software include: volumes of liquid to transfer; positions for pick up and positions for dispensing the liquids; positions where to pick up labware and positions where to set down labware; heat incubation times, etc.

As a first step in the methodology for the current assay or protocol, a filter card or other paper matrix having the dried blood spot (DBS) is punched to form the sample. Other biological samples such as saliva, tissue smears, urine or bacteria may be used in paper matrix applications, so the present methodology is described using DBS for reference purposes. The punch size and quantity of the samples may vary and may be punched, for example, in the range of 1 mm to 10 mm, using between 1 and 20 punches per well of a 96-well microtiter plate. A single 7.6 mm punch per well of a 96-well microtiter plate is optimal.

An alcohol is then added to each specimen. 30 μl of methanol is used for the current protocol, but the volume for spot treatment is preferably in the range of 20 μl to 60 μl and the useful range may generally extend from 10 μl to 350 μl. Furthermore, other alcohols may be used in place of methanol. Ethanol, Isopropanol, Isoamyle alcohol or other short carbon alcohols may be used.

The samples are then heated to cause the heme and other large proteins to bind to the filter paper such that they are prevented from going into solution during extraction and inhibiting later PCR reactions. Thus, the heme is fixed to the paper matrix prior to the extraction of the DNA. It should be noted that the fixing of protein to the matrix and the evaporation of the methanol are simultaneous and are in a single step.

The methanol is evaporated by placing the plate in the incubator at 110° C. for 30 minutes. The optimal temperature is 110° C., but the temperature range can be in the range of 50° C. to 120° C. The upper temperature is limited only by the ability to maintain a seal on each well of the microtiter plate and the melting temperature of the plate or seal material. Incubation time can vary depending on the incubation temperature.

100 μl of 30 mM Tris-HCl buffer is then preferably added to each specimen. This buffer is used to extract the DNA from the filter paper, provide a reagent concentration to prevent heme protein from coming out of the paper, and buffer the sample to the correct pH needed for later PCR reactions. The concentration of the buffer may range from 5 mM to 200 mM, preferably from 10 mM to 50 mM and optimally 30 mM. Furthermore, the pH range of the buffer may be any pH value that is suitable for the downstream nucleic acid application conditions and/or enzyme requirements. For example, the range for a PCR reaction would be the pH range optimal for the polymerase enzyme being used. This range would be from a pH of 5.5 to 9.5 for various polymerase enzymes. For the current PCR assay using a Klen-Taq Polymerase enzyme, a pH of 8.3 is optimal.

The plate is then sealed with a strong heat sealing device. The plate can be sealed with any sealing device or material that will produce and maintain a seal under the temperature and resulting pressure conditions created during nucleic acid extraction by heat treatment. Heating devices for extraction may include any device that will heat the microtiter plate to the desired temperature. Examples of such devices are an electrical heating block with a metallic plate adaptor, or an oven in which the plate would be placed.

The plate is heated again at 110° C. for 30 minutes. Again, the heating time and temperature may vary as above. This step releases the DNA into the tris-buffer without releasing excess heme protein which may inhibit PCR reactions. The resulting DNA extract is then ready for use in the desired assay.

The preferred methodology is summarized by the below example which is implemented by using a Beckman Coulter Biomek FX core robotic system.

### EXAMPLE

1. Punch a 7.6 mm blood spot into each well of a 96-well plate.

2. Add 30 μl MetOH (HPLC-grade methyl alcohol, methanol) to each specimen.

3. Heat the plate at 110° C. for 30 minutes to evaporate the methanol.

4. Add 100 μl of 30 mM Tris-HCl buffer pH 8.5 to each specimen.

5. Seal the plate with a strong heat sealing device.

6. Heat the plate again at 110° C. for 30 minutes.

1. A nucleic acid extraction method, comprising the steps of:
fixing a biological sample comprising DNA, a blood heme protein and other proteins to a paper matrix; and
allowing said biological sample to remain fixed to said paper matrix and simultaneously buffering the pH of said biological specimen, wherein DNA in said biological sample is extracted from said paper matrix without any said blood heme protein and other proteins coming out of said paper matrix.

2. The method of claim 1, wherein the step of fixing said biological sample includes heating said biological sample at 110° C. for 30 minutes.

3. The method of claim 1, wherein the step of allowing said biological sample to remain fixed includes adding 100 μl of 30 mM Tris-HCL buffer to said sample.

4. A nucleic acid extraction method, comprising the steps of:
punching a biological sample from filter paper into a well of a well plate to form a specimen;
adding an alcohol to said specimen;
evaporating said alcohol, wherein heme and other large proteins bind to said filter paper;
adding a reagent to said specimen, such that DNA is extracted from said filter paper while, simultaneously, said heme and said other proteins are prevented from coming out of said filter paper while said specimen is buffered;
sealing said plate; and
heating said plate, wherein said DNA is released into said buffer without releasing an excess of said heme and said other proteins such that a subsequent PCR reaction will not be inhibited.

5. The method of claim 4, wherein the step of adding said alcohol includes using an amount of said alcohol in the range of 10 μl to 350 μl.

6. The method of claim 5, wherein 30 μl of an HPLC-grade methyl alcohol is used.

7. The method of claim 4, wherein said alcohol is evaporated by placing said plate in an incubator at a temperature in the range of 50° C. to 120° C.

8. The method of claim 7, wherein said alcohol is evaporated at 110° C. for 30 minutes.

9. The method of claim 4, wherein said reagent added has a concentration in the range of 5 mM to 200 mM.

10. The method of claim 9, wherein 100 μl of a Tris-HCL buffer is added.

11. The method of claim 10, wherein said reagent has a pH in the range of 5.5 to 9.5.

12. The method of claim 11, wherein said reagent has a pH of 8.3.

13. The method of claim 4, wherein said plate is heated at 110° C. for 30 minutes after said plate is sealed.
